(19) United States
(12) Patent Application Publication
(10) Pub. No.: US 2025/0320738 A1
(43) Pub. Date: Oct. 16, 2025

(54) ADD ON UNIT FOR A POOL RELATED PLATFORM

(71) Applicant: Maytronics Ltd., Kibutz Yizrael (IL)

(72) Inventors: Alex Rachman, Kibutz Yizrael (IL); Boaz Ben Dov, Ram On (IL); Yair Hadari, Kibbutz Hulata (IL); Igor Lulko, Kibutz Yizrael (IL)

(73) Assignee: Maytronics Ltd., Kibutz Yizrael (IL)

(21) Appl. No.: 18/866,552

(22) PCT Filed: Apr. 26, 2023
(86) PCT No.: PCT/IB2023/054265

§ 371 Date: Nov. 17, 2024

(30) Foreign Application Priority Data
May 16, 2022 (IL) 293045
May 16, 2022 (IL) 293048

(51) Int. Cl. E04H 4/16; E04H 4/12; G08C 17/02

(52) U.S. Cl. CPC E04H 4/1654; E04H 4/1281; G08C 17/02

(57) ABSTRACT

There is provided an add on unit that includes a first communication unit that is configured to communicate with a pool related platform; a second communication unit that is configured to communicate with a computerized system; one or more additional components; a housing; and one or more adaptors that are configured to connect the add on unit to at least one out of the pool related platform or a cable that is connected to the pool related platform.

### CROSS REFERENCE

This application claims priority from IL patent application 293045 and from IL patent application 293048—both incorporated herein by their entirety.

### BACKGROUND

Millions of pool cleaning robots were sold in the last couple of years. Various pool cleaning robots have a fixed hardware configuration that cannot be upgraded. For example—a pool cleaning sensor is sold with a certain set of sensors—and there is no effective way to add other sensors. Yet for another example—some pool cleaning robots may communicate only over a cable communication—and through an external power source.

There is a growing need to provide an effective upgrade solution to pool cleaning robots.

### SUMMARY

There may be provided an add on unit for a pool related platform (PRP).

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 illustrates an example of an add on unit;

FIG. 2 illustrates an example of an add on unit and its environment;

FIG. 3 illustrates an example of pool cleaning robot and an add on unit;

FIGS. 4-9 illustrates an example of at least one part of a filtering unit of pool cleaning robot and an add on unit;

FIG. 10 illustrates an example of a floating unit, a pool cleaning robot and an add on unit;

FIGS. 11-12 illustrate examples of a cable and an add on unit;

FIG. 13 is an example of a method; and

FIGS. 14-38 illustrate examples of add on units and/or add on unit parts.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DESCRIPTION OF THE PREFERRED EMBODIMENTS

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The subject matter regarded as the invention is particularly pointed out and distinctly claimed in the Summary of the invention of the specification.

The invention, however, both as to organization and method of operation, together with objects, features, and advantages thereof, may best be understood by reference to the following detailed description when read with the accompanying drawings.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

The term “and/or” is additionally or alternatively.

The terms “control unit”, “remote control device”, “control device” and “mobile computer” are used in an interchangeable manner.

Any reference in the specification to a pool cleaning robot should be applied mutatis mutandis to a method that can be executed by the pool cleaning robot and to a computer readable medium that stores instructions to be executed by the pool cleaning robot.

Any reference in the specification to a mobile computer should be applied mutatis mutandis to a method that can be executed by the mobile computer and to a computer readable medium that stores instructions to be executed by the mobile computer.

Any reference in the specification to method should be applied mutatis mutandis to a pool cleaning robot and/or to a mobile computer that execute the method and/or to a computer readable medium that stores instructions that once executed result in an execution of the method.

Any reference in the specification to a non-transitory computer readable medium should be applied mutatis mutandis to a pool cleaning robot and/or a mobile computer capable of executing the instructions stored in the non-transitory computer readable medium and should be applied mutatis mutandis to method that may be executed by a pool cleaning robot and/or a mobile computer that reads the instructions stored in the non-transitory computer readable medium.

Because the illustrated embodiments of the present invention may for the most part, be implemented using electronic components, touch screens and circuits known to those skilled in the art, details will not be explained in any greater extent than that considered necessary as illustrated above, for the understanding and appreciation of the underlying concepts of the present invention and in order not to obfuscate or distract from the teachings of the present invention.

Any reference in the specification to a method should be applied mutatis mutandis to a system capable of executing the method and should be applied mutatis mutandis to a non-transitory computer readable medium that stores instructions that once executed by a computer result in the execution of the method.

There may be provided an add on unit that can be easily installed by a user of a Pool related platform (PRP). It may be plug and play installed—and may not reduce the PRP performance. The add on unit may be in communication (wireless or not) with the PRP and an remote computerized system such as a cloud computing environment. The installation of the add on unit may not require an sealing\screws on the PRP and/or the add on unit.

The PRP may be any platform that may perform an operation related to a fluid of a pool-cleaning, changing chemical composition, monitoring, and the like. Examples of a PRP include a pool robot that differs from a PCR, a PCR, a floating unit, a skimmer, and the like. Any example related a PCR may be applied mutatis mutandis, to any other PRP.

The add on unit may be used to upgrade the PRP—for example provide more abilities such as water quality sensing, drowning alert, wireless communications, navigation and more. The add-on unit may be configured to be connected to any part of the PRP—for example—to the outside or inside of the PRP, on top or at the bottom or the sides of the PRP, to any PRP related parts such as cable or a swivel, on motor unit, on a back pulley, on a sidewall of a filtering unit, on top of the filtering unit.

The add on unit may include a power source such as one or more batteries. The batteries can be located inside the add on unit but could also be located outside of it—may be connected to its housing. In other embodiments the add on unit connected to outside power source (for example batteries of the PRP).

The add on unit may be provided with sensors and\or other additional components that are configured to upgrade and/or enhance the functional capabilities and/or abilities of the PRP. Examples to one or more additional components may include a navigation unit, a drown detection unit, a processor (for example Central Processing Unit). The PRP may also include one or more communication units.

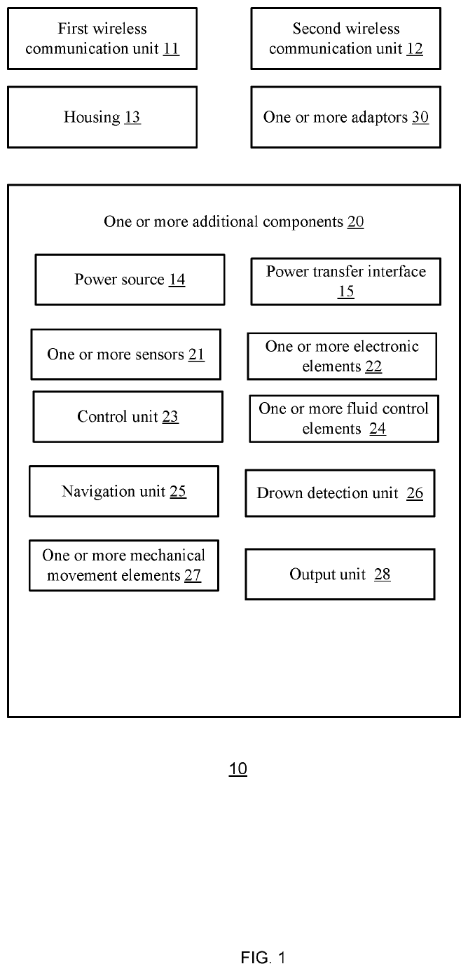

FIG. 1 illustrates an example of an add on unit 10 that may include a first wireless communication unit 11 that is configured to wirelessly communicate with a pool related platform (PRP), a second wireless communication unit 12 that is configured to wirelessly communicate with a remote computerized system (such as an external power source, a relay, a cloud computing environment, a server, a mobile phone, a control unit, a pool control system, a pool chemical controller, a fluid management system, a pump controller, a chlorinator, and the like), one or more additional components 20, a housing 13 (may be a sealed housing or an unsealed housing), and one or more adaptors (collectively denoted 30) that are configured to connect the add on unit to the pool related platform and/or to a cable that is connected to the PRP.

The first wireless communication unit 11 may communicate over a relatively short communication path (for example between 1-40 centimeters), while the second wireless communication unit 12 may communicate over a longer communication path (for example—may exceed 1, 2, 5, 10, 15, 50, 100, 200 meters, and even more). Any wireless communication protocol may be used-for example BLE™, Bluetooth™, and the like. Non-limiting examples of communication schemes are illustrated in U.S. provisional patent Ser. No. 63/265,937 filing date Dec. 22, 2022—which is incorporated herein by reference.

FIG. 1 illustrates the add on unit 10 as including a power source 14. It should be noted that the power source may be external to the add on unit and/or that the add on unit may include a power transfer interface 15 for receiving power (for example for powering the add on unit 10 and/or for charging power source 14. The power transfer interface may be a contactless power transfer interface (for example—inductive charging) and/or a cable based power transfer interface.

The one or more adaptors 30 may be configured to detachably connect the add on unit to the PRP and/or the cable.

The one or more adaptors 30 may be self-connectable to PRP and/or the cable—thus the one or more adaptors may connect the add on unit 10 without assistance from the PRP and/or the cable and/or without modifying the PRP and/or the cable. For example-the one or more adapters may not need to be fastened by screws to the PRP and/or the cable.

The one or more adaptors may be of one or more types such as mechanical adaptors and/or magnetic adaptors and/or electrostatic adaptors. For example-they may include one or more cable clip adaptors, one or more rotation-based adaptors, one or more sliding adaptors, and the like.

One or more add on units may be shaped and size to be attached to one or more parts of the PRP. Different add on units may be shaped and sized to be attached to different parts of the PRP. It should be noted that an add on unit may be shaped and sized to be attached to more than a single part of the PRP. An add on unit may be shaped and sized to be located within a part of the PRP, partially within a part of the PRP, outside the part of the PRP, and the like. The PRP may be a pool cleaning robot (PRC) and non-limiting examples of parts of a PRC include a housing, a filtering unit, a pump motor, a drive motor, an impeller, an inner housing of the PRC, a cleaning element of the PRC, a power supply unit of the PCR, a jet propulsion system, any other propulsion system, an electronic module of the PRC, and/or any other part of the PCR. Non-limiting example of PRCs and their parts are provided in U.S. Pat. Nos. 9,410,338, 10,533,335, 10,843,106, 10,458,139, 11,124,982, 10,858,852, 10,982,456—which are incorporated herein by reference.

The one or more additional components 20 may be self-operable, may be a part of a unit that is (at least) partially located outside the PRP, may communicate and/or cooperate with a unit that is located outside the PRP, and the like.

Non-limiting example of the one or more additional components may include one or more sensors 21, and/or one or more electronic elements 22 other than one or more sensors, control unit 23, one or more fluid control elements 24 for controlling a parameter of a fluid of a pool (for example may output one or more chemical agents for changing a chemical parameter of the fluid of the pool-such as pH level, alkalinity of fluid, chlorine level, or any other chemical parameter), and the like.

The one or more sensors may be of any type and/or may be used for any purpose—for example the one or more sensors may be chemical sensors, biological sensors, radiation based sensors (any radiation—light, thermal radiation, infrared, near infrared), cameras, image sensors, non-image sensors, temperature sensors, acoustic sensors, passive sensors, active sensors (LIDAR, radar), mechanical sensors (vibration sensor, velocity sensor, accelerometer, gyroscope), location sensors (GPS, magnetic based), barometer, spectrometer, and the like. The purposes may include, for example, navigation, turbidity sensing, drown detection, water quality monitoring, water quality control.

Examples of chemical and/or biological sensors of a PCR are illustrated in US patent applications publication 2018/0266131—which is incorporated herein by reference. Chemical and/or biological sensors may provide information regarding Chlorine, Total Dissolved Salts (TDS), Turbidity, Phosphates, Temperature, pH, ORP, Flow Rate, Algae, Bacteria, circulated fluid flow rates in the filtering system, and any or all other physical, chemical and biological parameters or species, and the like.

The one or more sensing elements of the one or more sensors may be kept in certain conditions—for example may be kept dry, wet, always wet, always dry, and the like. Wet may be fully submerged in fluid, partially submerged in fluid, within a humid environment, and the like.

The one or more additional components may include, for example, a navigation unit 25 or at least include one or more sensors that belong to the navigation unit.

The one or more additional components may include, for example, a drown detection unit 26 or at least include one or more sensors that belong to the drown detection unit.

The one or more additional components may include, for example, one or more mechanical movement elements 27 for moving at least one other components (for example another additional elements) such as a sensor—for example—in and out of the housing 13 (for example for contacting fluid and then for retreating [via a selectively covered opening of the housing] into the housing 13).

The one or more additional components may include, for example, output unit 28 for outputting items (for example—a float that is detachable and can be connected to a submerged element).

While FIG. 1 illustrates one or more sensors 21, one or more electronic elements 22, one or more control units 23, one or more fluid control elements 24, navigation unit 25, drown detection unit 26, and one or more mechanical movement elements 27, and output unit 28—but it should be noted that an add on unit may include only one, only some or all of these one or more additional components.

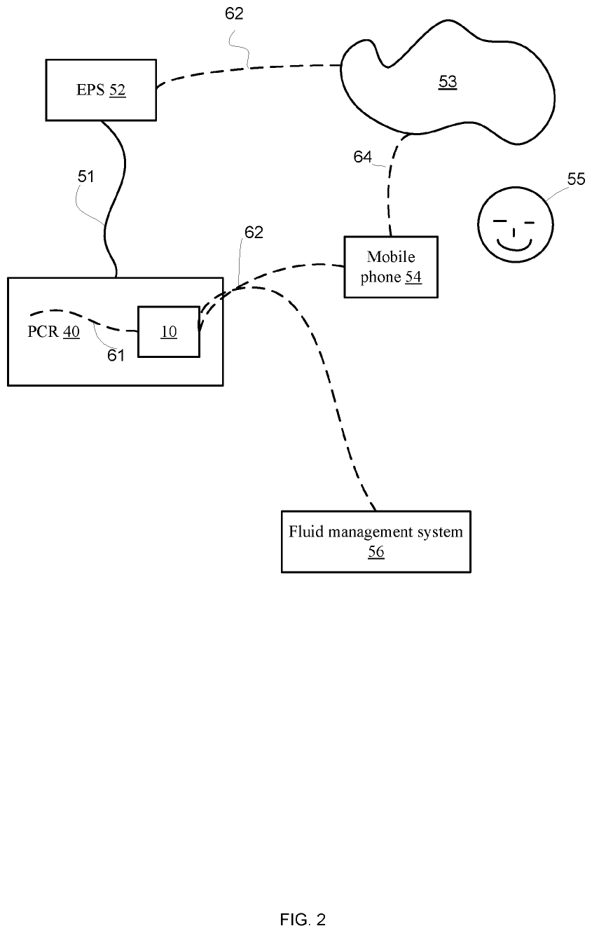

FIG. 2 is an example of a communication scheme between PCR 40, add on unit 10, cable 51, external power supply 52, cloud computing environment 53, mobile phone 54, user 55, and fluid management system 56.

The PCR 40 communicated via cable 51 with external power supply (EPS) 52 that in turn communicates via EPS wireless path 63 with cloud computing environment 53. The cloud computing environment 53 communicates via cloud to mobile wireless path 64 with mobile device 54 of user 55. The add on unit 10 communicated with PCR 40 over first wireless path 61 and communicates with mobile phone 54 and/or fluid management system 56 via one or more wireless paths such as second wireless path 62.

A communication may include at least one of receiving and/or transmitting information and/or commands and/or requests and the like. Information may include status information or other information.

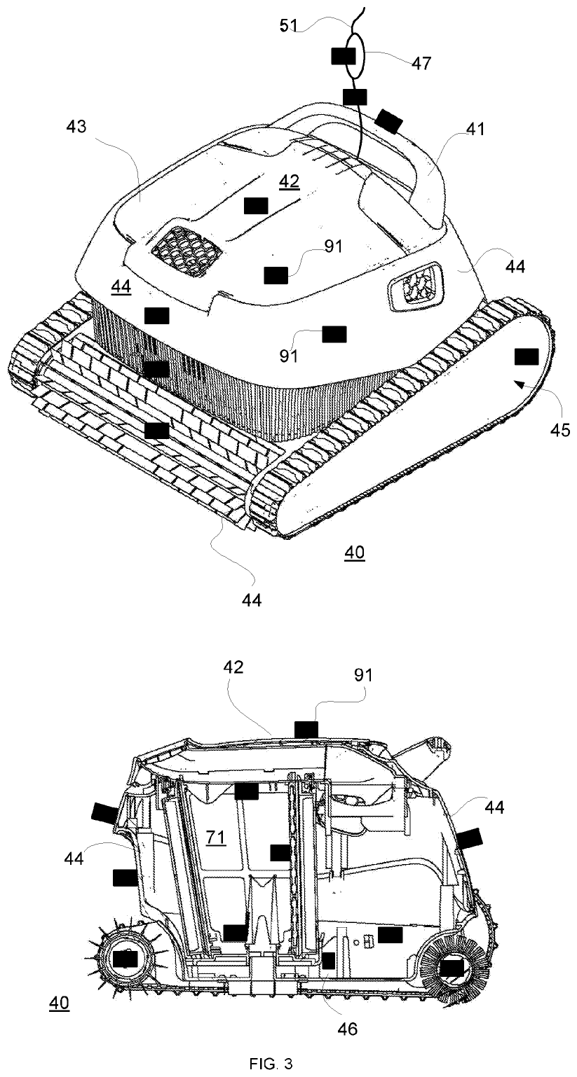

FIG. 3 illustrate examples of positions (collectively denoted 91—represented by black boxes) of the add-on unit 10. On handle 41, on the top 42 of PCR housing 43, on one or more sidewalls 44 (rear and/or front and/or left and/or right) of PCR housing 43, within (for example within an axis or rotation of or surrounding the axis or rotation or connected to the axis of rotation of) one or more cleaning elements such as brushes 45, within filtering unit 71, within a pump motor 46, connected to cable 51, connected to swivel 47, and the like.

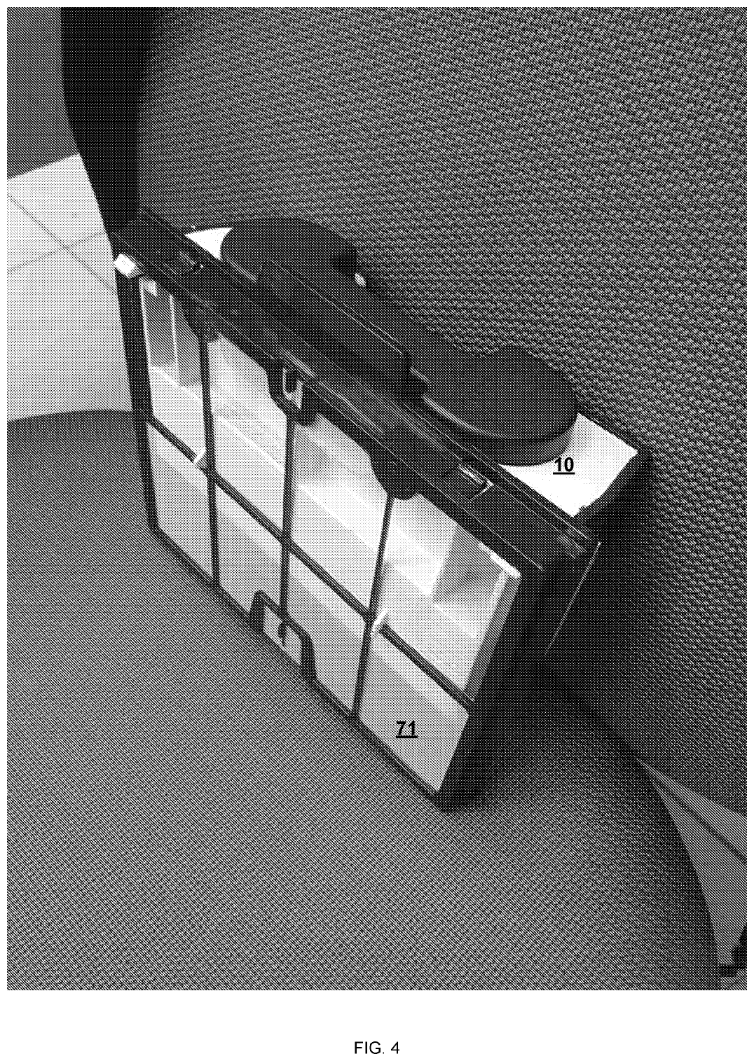

FIG. 4 illustrates an example of an add on unit 10 that is connected to a sidewall of filtering unit 71. Other parts of the filtering unit are not shown.

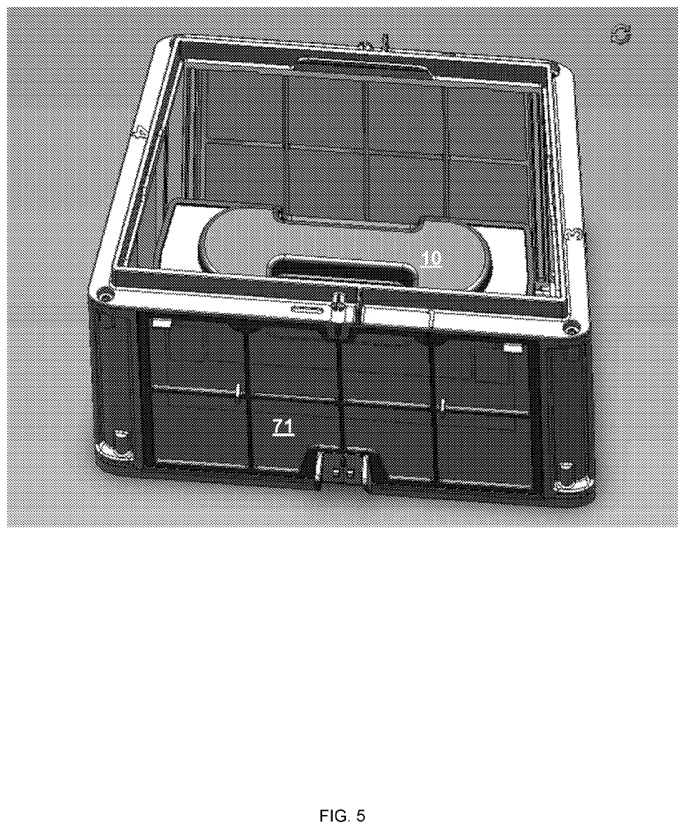

FIG. 5 illustrates an example of an add on unit 10 that is located within the filtering unit—and on top of the bottom of filtering unit 71.

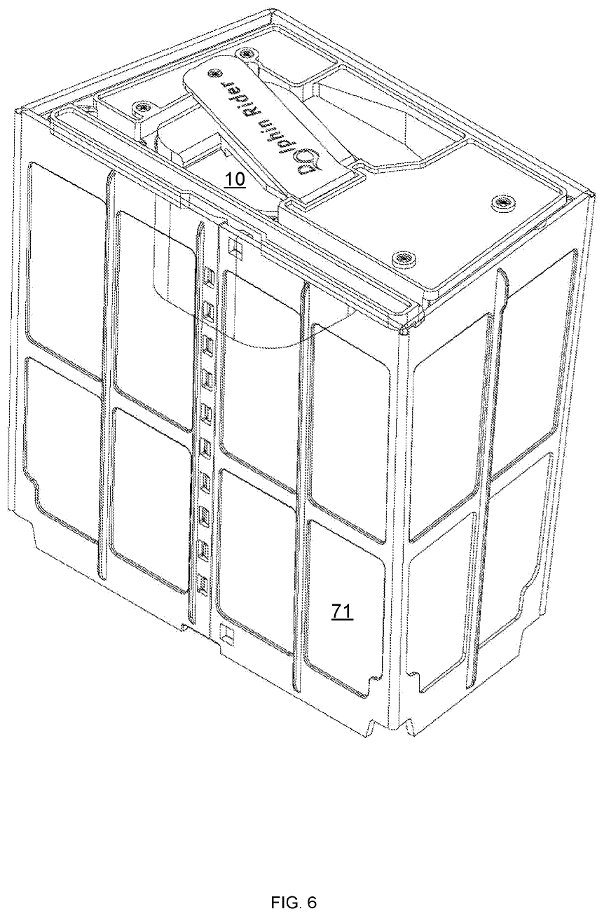

FIG. 6 illustrates an example of an add on unit 10 that is inserted into the filtering unit—and is connected to a sidewall of filtering unit 71.

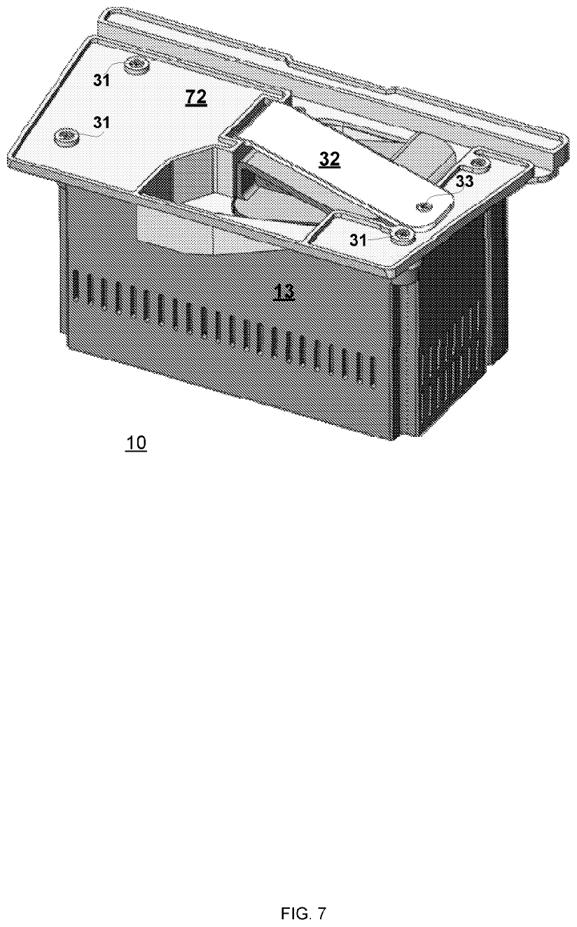

FIG. 7 illustrates an example of an add on unit 10 that is connected to a cover 72 of a filtering unit. In FIG. 7 the housing 13 of add on unit 10 is screwed to the cover 72 (see screws 31), and there is a top adaptor 32 that is screwed (see screws 33) to the cover 72. It should be noted that the connectivity may be without using screws—for example may include magnetic attachments, or other mechanical adaptors.

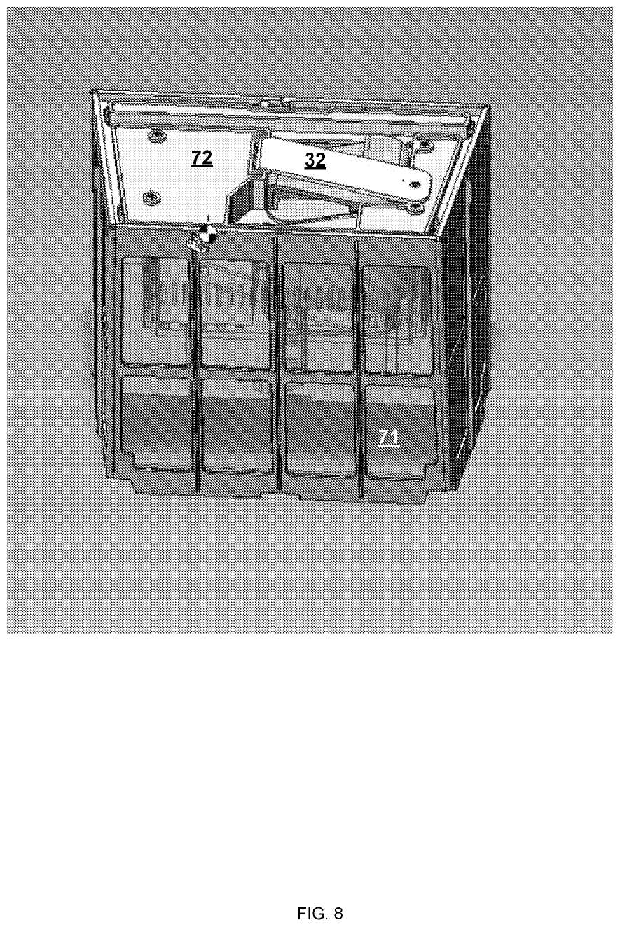

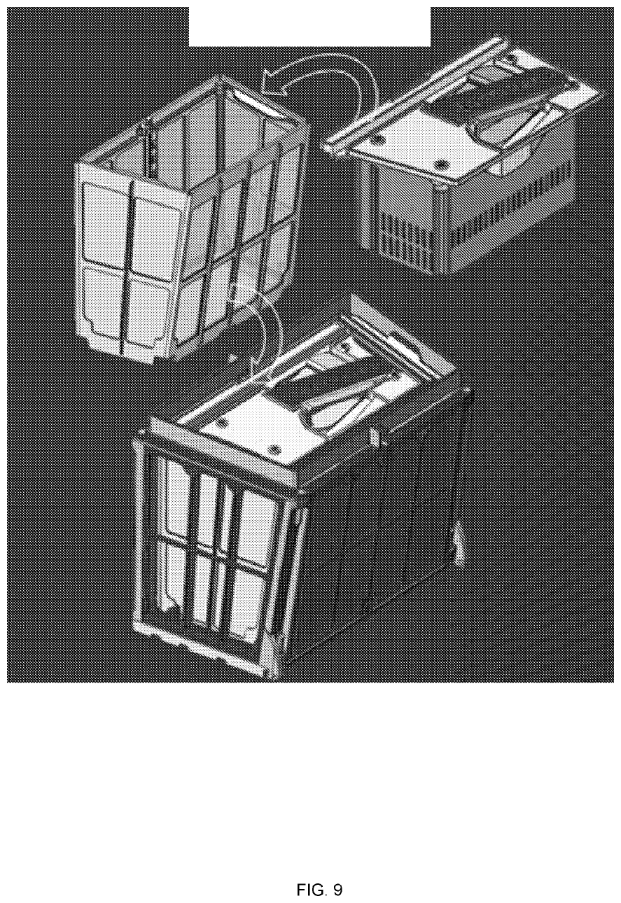

FIGS. 8 and 9 illustrate the add on unit 10 of FIG. 7 and the filtering unit 71.

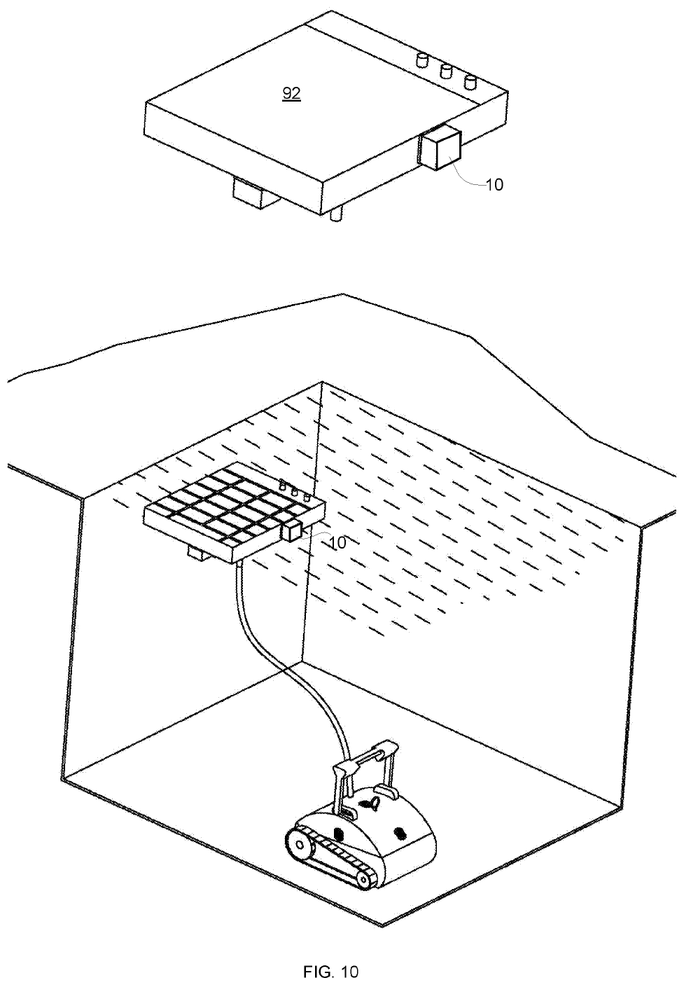

FIG. 10 illustrates a PRP that is a floating unit 95 that is configured to provide power (for example from solar panels or any other source) to PCR 40. The floating unit 95 may include sensors and/or water control elements, and/or communication units. The add on unit 10 is located at the side of the floating unit—but may be attached to any other part of the floating unit.

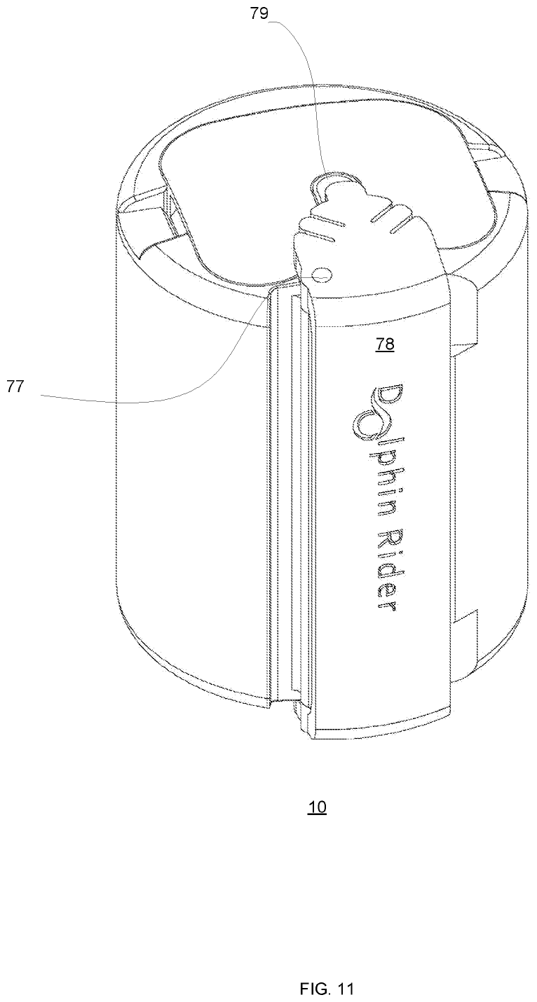

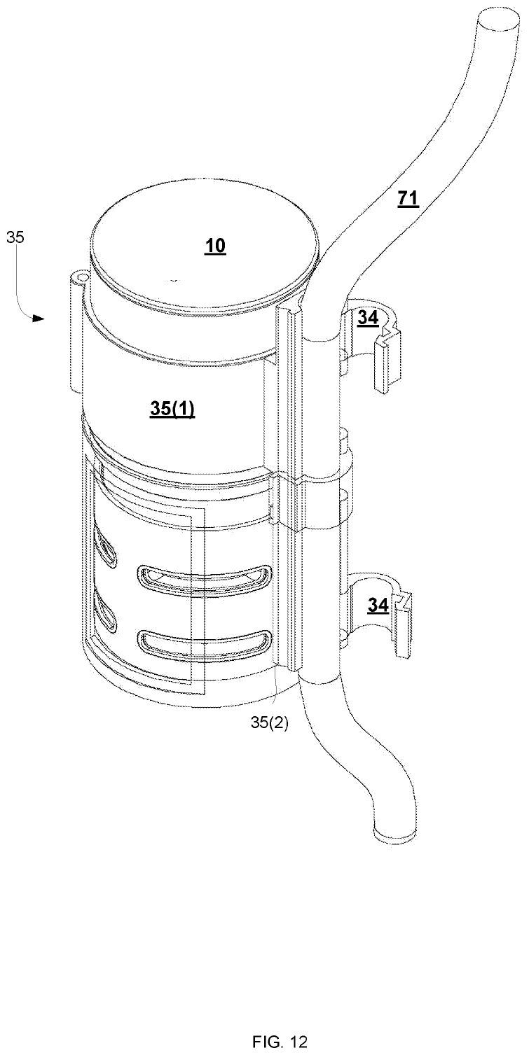

FIGS. 11-12 illustrate examples of add on units 10 that are connected to a cable 51.

In FIG. 11 the add on unit 10 included a cable opening 79 through which a cable may be inserted. There is a movable (for example a rotatable) interface 78 that rotates about axis 77 and is configured to allow (when in open position—for example when it rotates clockwise on relation to the closed position of FIG. 11) the cable to be inserted on the cable opening. The interface (when in a close position) may press against the cable (when inserted in to the cable opening 79) and fasten the add on unit to the cable.

In FIG. 12 there is a cable opening 79 that may be fastened in any manner to a floating unit 401 that may move in relation to another part 402 of the add on unit that may include any of the components of FIG. 1. The other has an opening 403 for receiving fluid and the float may selectively close and open the opening—by changing the position of the opening closing element 404.

In FIG. 12, the add on unit 10 is illustrated as including adaptors such as clamps 34 and adaptor 35 that has a first part 35 (1) that surrounds the add on unit and a second part 35 (2) that is connected to the clamps 34 and to the first part. The clamps 34 hold cable 71. There may be provided a method for operating an add on unit.

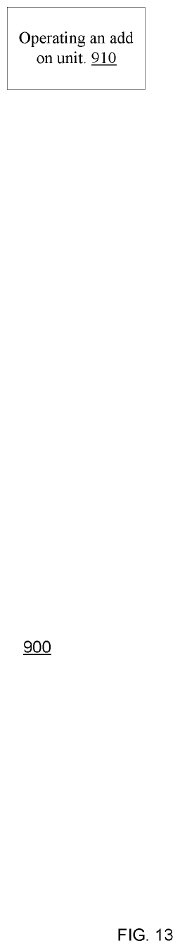

FIG. 13 illustrates an example of method 900 for operating an add on unit. Method 900 may include step 910 of operating an add on unit.

Step 910 may include operating any of the add on units and/or any of the units (of any of the add on units) and/or components (of any of the add on units) illustrated in any of the figures (for example any one of FIGS. 1-12, and/or 14-38)—for example communicating, performing measurements, supplying power, receiving power, controlling any of the units and/or components illustrated in any of the figures, protecting the sensors, maintaining sensor probes and the like.

### Always Wet (Protecting Sensors)

While some of the examples refer to an add on unit—it should be noted that the add on unit is an example of a sensor protective system that may differ from an add on unit—for example may be an integral part of the PRP, may be included in a PRP when distributed to the client, and the like.

Some sensors may require that their sensing regions are maintained wet—either by being submerged in fluid or by being within a humid environment (of at least a predefined humidity). In some cases the sensing regions may be within a dry environment—but just for up to a predefined period of time (for example of a few minutes, a few hours and the like).

It should be noted that in some cases the sensing regions should be maintained wet only when activated—thus when idle the sensing regions are not damaged due to dryness—or have their own protection measures.

There may be provide an add on unit that may include one or more sensing regions for sensing one or more parameters of a fluid—at least when in contact with the fluid. The add on unit may also include a protection unit that is configured to prevent the one or more sensing regions from being damaged due to dryness.

The protection may be obtained in at least one of the following manners:

a. Stopping measurements of at least one of the one or more parameters of the fluid.

b. Preventing measurements of at least one of the one or more parameters of the fluid.

c. Maintaining the one or more sensing regions within fluid and/or humid.

Any one of measures (a), (b) or (c) may be triggered when detecting a triggering event.

The protection unit may be configured to stop a measurement of at least one of the one or more parameters of the fluid when detecting a triggering event that indicates that a completion of the measurement will introduce a dryness related damage.

The protection unit may be configured to perform a protective step (for example—measure (a) and/or measure (b) and/or measure (c)—and/or any other measure) following a detection of a triggering event.

The triggering event may be an extraction of the add on unit from the fluid.

The triggering event may include having the add on unit tilted at an angle that is outside a predefined angular range.

The detection of the triggering event may be based on acceleration signals and/or tilt angle signals and/or sensing an environment of the add on unit.

The add on unit may include a contactless fluid sensor that is configured to sense a wetness of the environment without contacting the environment. The contactless fluid sensor may be protected from the fluid—and this may lengthen the lifespan of the contactless sensor.

The protection unit may perform any protective step when sensing and/or estimating and/or determining that a damaging event may occur—for example that the sensing regions may be exposed to a dry environment and/or that a completion of the measurement will introduce a dryness related damage—for example when being (the add on unit and/or a pool related platform and/or a cable associated with the add on unit) are at least partially extracted from a pool and/or when the add on unit is tilted to a degree that may drain the add on unit from fluid (usually when being close to the water line or being above the waterline).

The sensing and/or estimating and/or determining may use machine learning, may be based on previous events and previous sensor readings indicative of events, may not be based on machine learning, and the like.

The sensing and/or estimating and/or determining may be based on outputs from one or more sensors such as an accelerometer, a tile angle sensor (for example—sensing when an add on unit is being tilted at an angle that is outside a predefined angular range), an environmental sensor—for example a contact based or contactless fluid sensor for sensing when the add on unit is extracted from the pool.

Any of measures (a), (b) or (c) should be completed before the starting of the damaging event. For example—once the add on unit is extracted from a pool—it may be maintained outside the pool for an unknown duration. Thus, for example, measure (c) should be completed before the extraction of the add on unit from the pool.

The execution of measure (c) may require a certain time period. Measure (c) may be triggered at least said certain time period before the extraction of the add on unit from the pool.

The one or more sensing regions may be located within one or more spaces of the add on unit. A space may be defined by the add on unit—or may be partially defined by the add on unit.

Measure (c) may be applied for any duration—for example during a few seconds or minutes in which the add on unit is out of a pool—and even till long storage periods (even months) in which the add on unit (and the pool related platform) are stored (for example during the winter). Once a trigger event is detected—the protective step may be executed (continuously) till there is an indication that it may be stopped. For example—an inner space in which the sensing regions should be kept wet—may be closed for any duration.

Regarding measure (c)—it may include at least one out of:
a. Maintaining at least a predefined humidity within the one or more spaces.
b. Maintaining at least a predefined amount of fluid within the one or more spaces.
c. Maintain the one or more sensing regions within fluid captured within the one or more space.
d. Controlling a flow of fluid from the one or more spaces.
e. Controlling a flow of fluid to the one or more spaces.
f. Controlling, by one or more flow control elements, a flow of fluid between the one or more spaces and an environment (may be a location outside the add on unit) of the one or more spaces.
g. Controlling the flow of fluid using a mechanism.
h. Controlling the flow of fluid using one or more pumps.
i. Controlling the flow of fluid using one or more floating units.
j. Controlling the flow of fluid using one or more motors.
k. Controlling the flow of fluid through one or more fluid openings of the one or more spaces.
l. Controlling the flow of fluid through one or more fluid openings using one or more movable seals that are configured to selectively seal the one or more fluid openings.
m. Controlling the flow of fluid through one or more fluid openings using one or more fluid openings closing elements.
n. Controlling the flow of fluid through one or more fluid openings using one or more apertured elements that are configured to be moved between (a) an open position in which apertures of the apertured elements are at least partially aligned with the one or more fluid openings, and (b) a closed position in which apertures of the apertured elements misaligned with the one or more fluid openings and the apertured elements and block a passage of fluid between the one or more spaces and an environment of the one or more spaces.

The sensor protective system may include one or more adaptors that are configured to connect the sensor protective system to at least one out of a pool related platform or a cable that is connected to the pool related platform.

The one or more adaptors may be configured to detachably connect the sensor protective system to the at least one out of the pool related platform or the cable.

The one or more adaptors may be self-connectable to the at least one out of the pool related platform or the cable.

The one or more adaptors may include one or more cable clip adaptors.

The sensor protective system may include a power supply.

The sensor protective system may be shaped and sized to be at least partially inserted in a filtering unit of the pool related platform.

The sensor protective system may be shaped and sized to be located within a housing of the pool related platform and to be connected to a filtering unit of the pool related platform.

The one or more sensors may include a water quality sensor.

The sensor protective system may be an add on unit.

The sensor protective system may differ from an add on unit.

There may be provided a method for operating an sensor protective system of any one of the preceding claims.

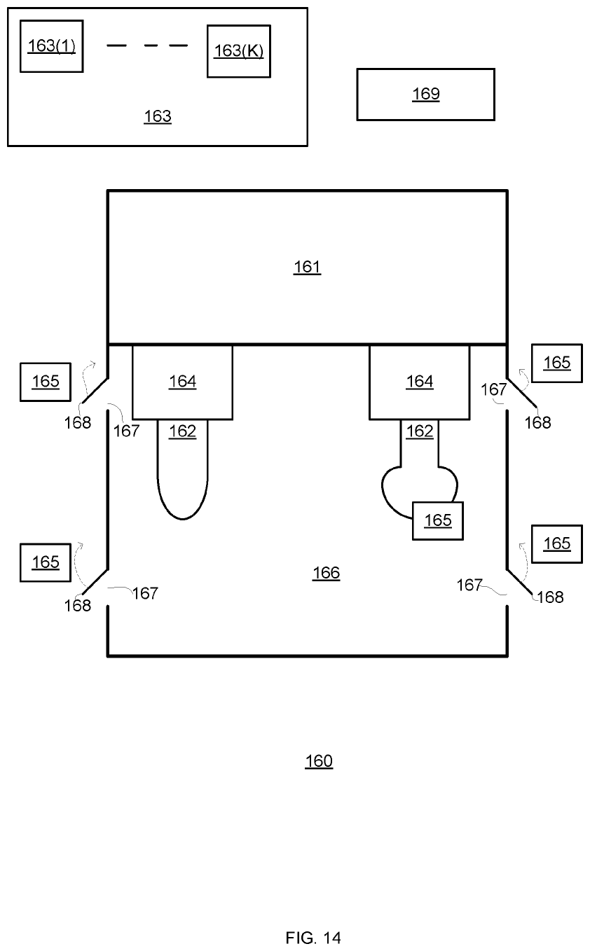

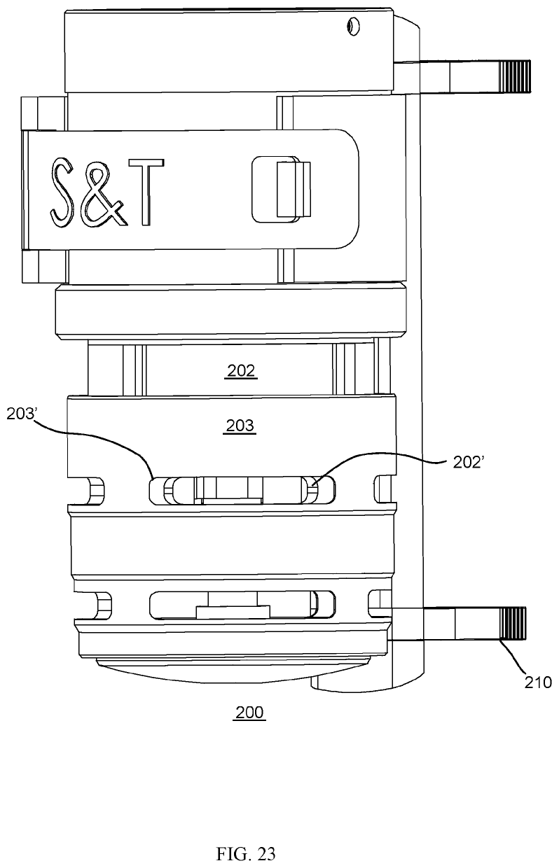

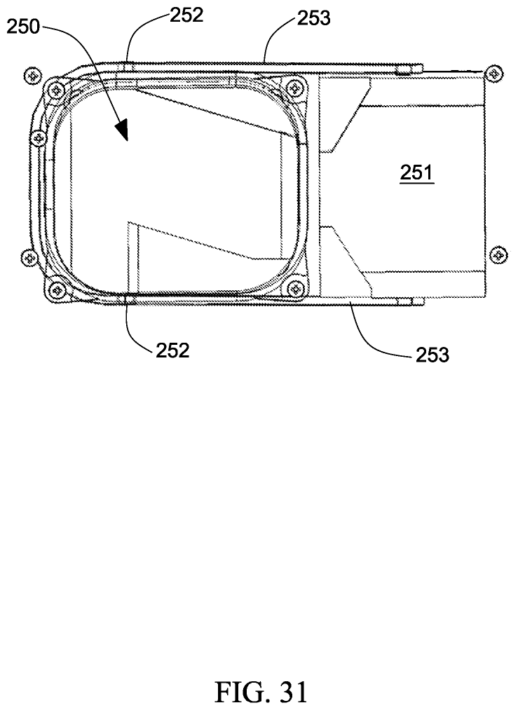

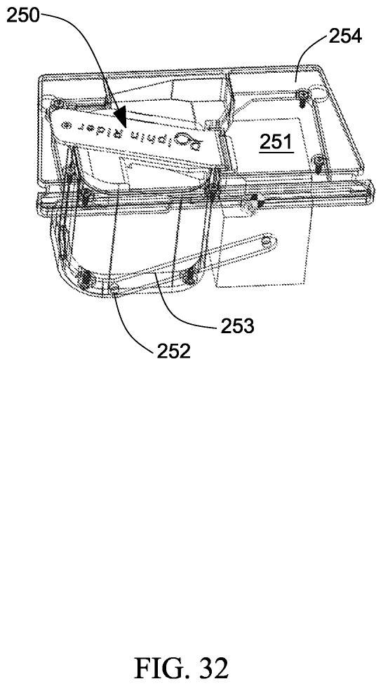

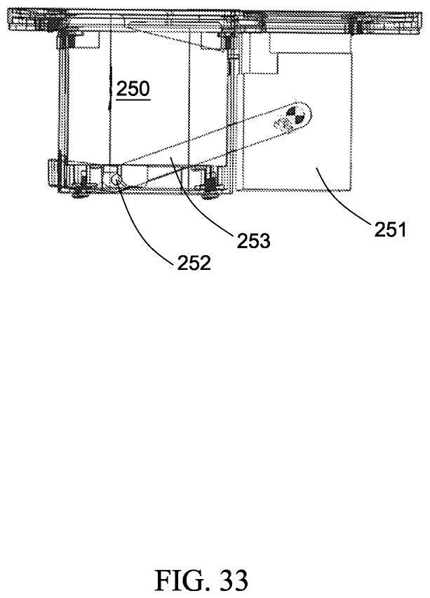

FIGS. 14-33 illustrate examples of add on units that are configured to implement at least measure (c). Each one of the add on units may apply measure (a) and/or measure (b).

FIGS. 14-33 illustrates add on units that include one or more sensors (collectively denoted 63) that are used for sensing a trigger event that may indicate that a protective measure should be taken. For example the triggering event may be indicative that damaging event may occur (for performing any one of measures (a), (b) or (c)), a control unit 69 for controlling the add on units (for example performing the estimating and/or determining that a damaging event may occur and/or responding to the estimation and/or determining).

FIG. 14 illustrates that there may be multiple (K) sensors 61(l)-63(K). At least some of the sensors may be sued for sensing a triggering event. These sensors may include at least one accelerometer, at least one environmental sensor (for example a contactless sensor for sensing the presence of fluid), at least one tilt angle sensor, and the like.

FIGS. 14-21 illustrate two sensors that include a base 64 and sensing regions 62 that are protected against being damaged due to dryness. The sensing regions 62 are located within sensors inner space 66 of the add on unit having fluid openings 67.

In FIGS. 14-20 the protection unit includes flow control units that may include fluid opening closing units 68 movable by motion elements 65 such as motors, floating units and the like, for selectively opening or closing the fluid openings 67. In FIGS. 14-20 there are four fluid openings 67—but there may be any number of fluid openings 67.

FIGS. 14-21 also illustrate another space 61 in which electronics and/or other parts of a sensing unit are located.

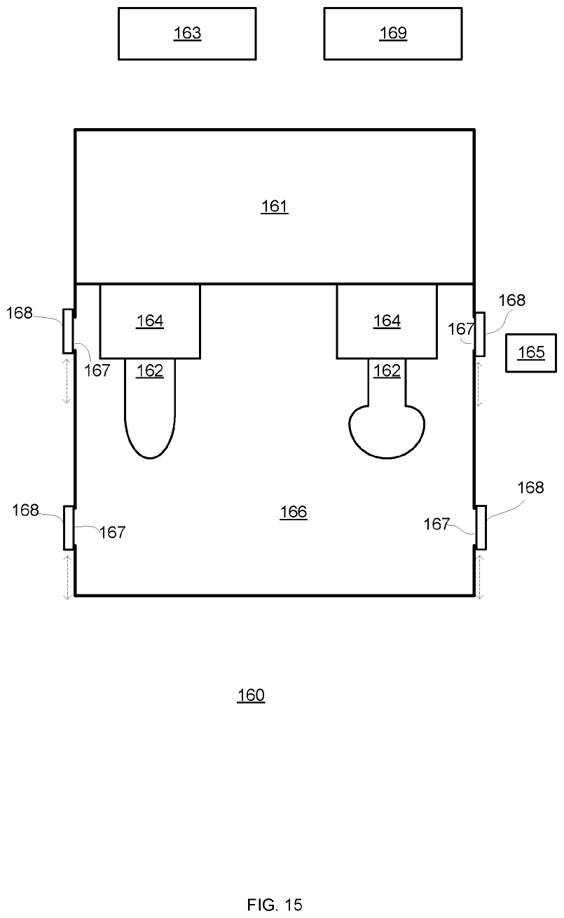

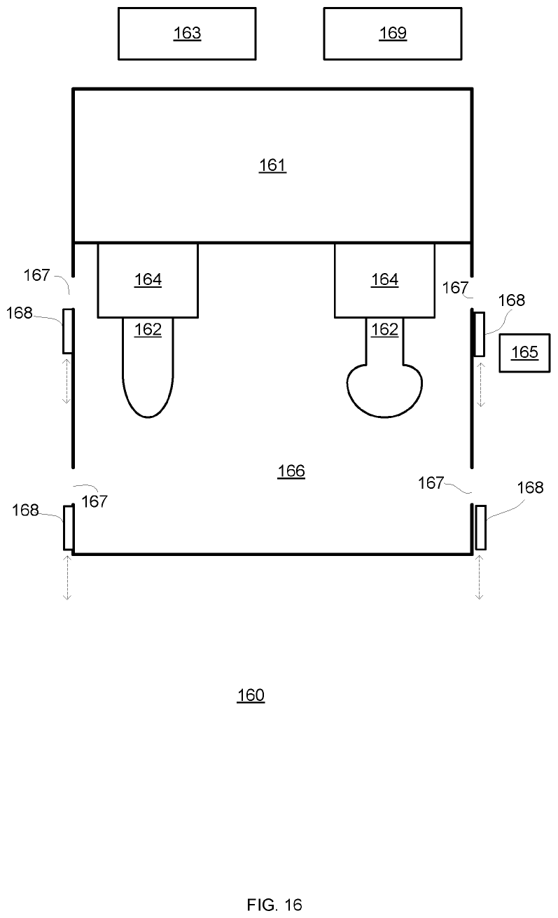

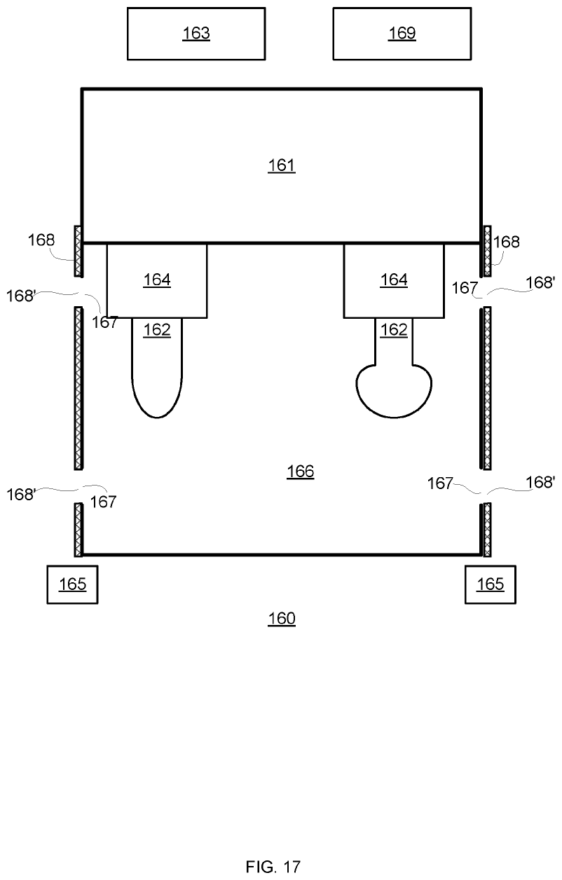

FIG. 14 illustrates a motion element 65 per fluid opening closing unit 68. FIGS. 15-16 illustrate a single motor element 65. FIGS. 17-20 illustrate two motor elements 85. This may be for simplicity of explanation. A motor element may use to control the movement of one or more fluid opening closing unit. One or more fluid opening closing units may be moved independently from each other—or may be moved together.

The one or more fluid opening closing units may be moved in any manner-rotation (see FIG. 11), linear (for example up and down movement—see FIGS. 15-21), and the like.

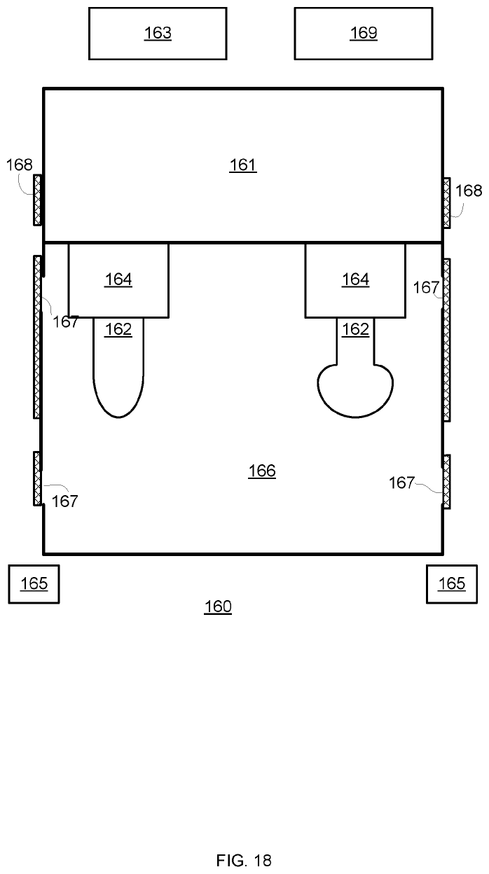

FIGS. 14, 16, 17, 19 and 20 illustrate the fluid opening closing unit 68 in an open position. FIGS. 15 and 18 illustrate the fluid opening closing unit 68 in a closed position.

FIGS. 15-20 illustrate the fluid opening closing unit 68 as apertured elements having openings 68′—whereas when in an open position the openings 68′ are aligned with the fluid openings 67, and when in closed position the openings 68′ are misaligned with the fluid openings 67. In FIGS. 15-18 the apertured elements move upwards and downwards.

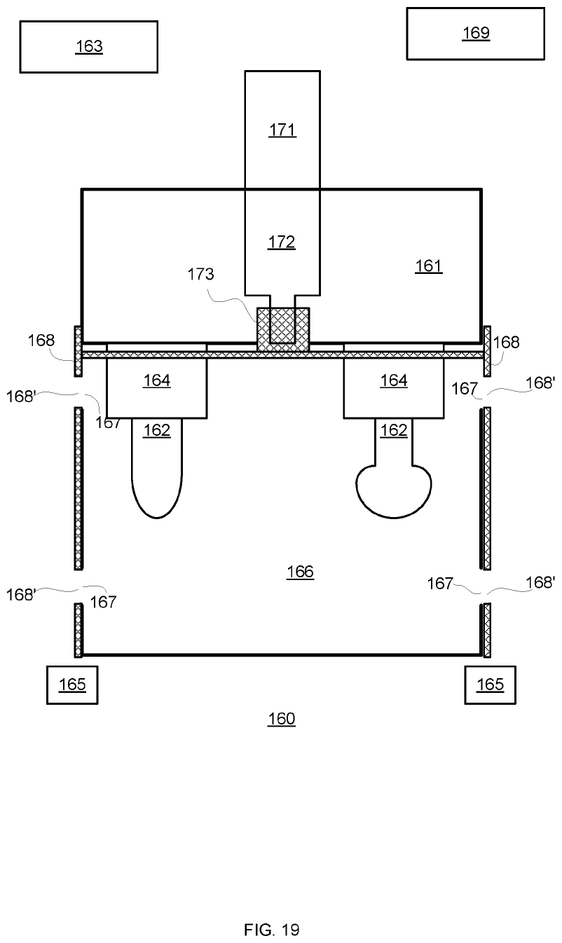

In FIG. 19 the add on unit includes a motor 17 that is configured to rotate a rotatable element (such as an axis), wherein a gear 63 converts the rotational movement to a z-axis movement—thereby moving the apertured elements upwards (when rotating along a first direction) and downwards (when rotating along a second direction opposite to the first direction).

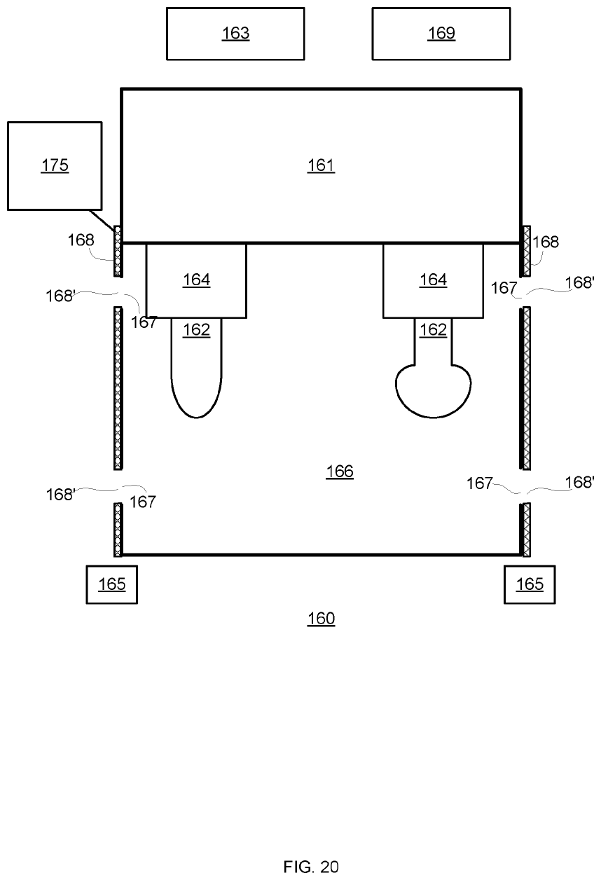

In FIG. 20 the apertured elements are moved by a flow control unit that is a floating unit 75—so that when the level of fluid in the environment of add on unit decreases—the fluid openings 67 are closed—and when the level of fluid in the environment of add on unit increases—the fluid openings 67 are opened.

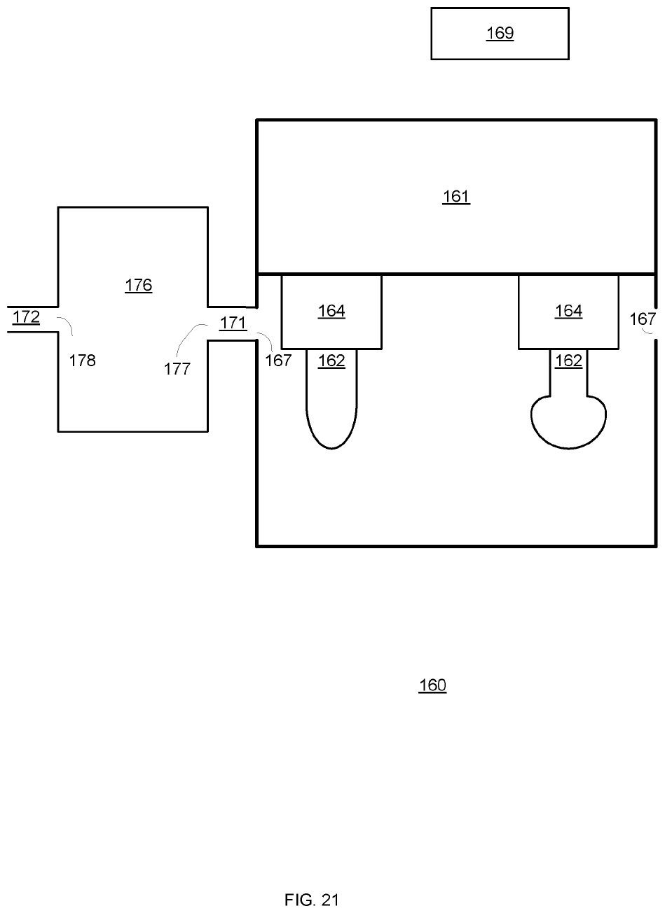

FIG. 21 illustrates a flow control unit.

FIG. 14 illustrates an add on unit 60 that includes two fluid openings 67—but there may be any number of fluid openings 67.

One fluid opening is open. The second fluid opening is in fluid communication with a first port 761 of pump 76 via first fluid conduit 771. A second port 762 of pump 76 is in fluid communication with a second fluid 772. The pump may be of any type—for example a membrane pump or another type of pump. The pump may suck fluid and allow fluid to enter the sensors space or may drain the fluid or may stop the flow of fluid by sealing the flow of fluid through the pump—76.

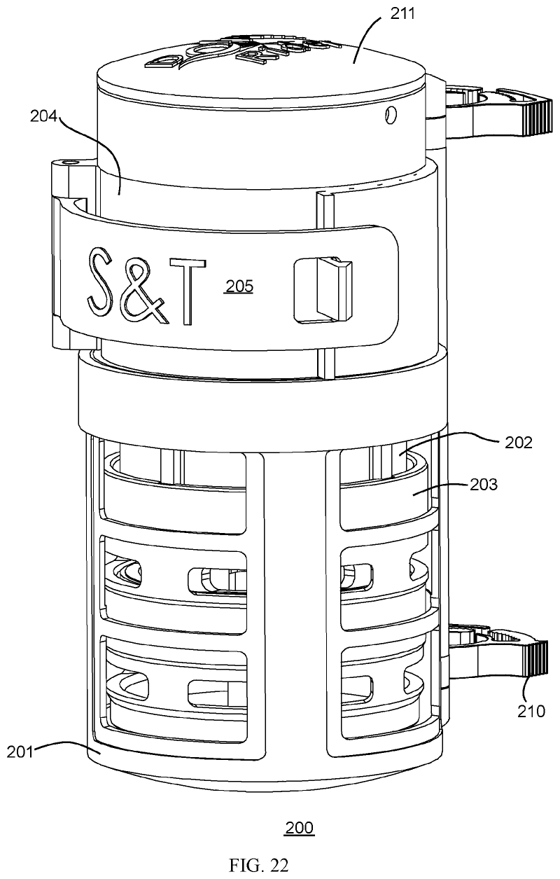

FIGS. 22-38 illustrates examples of add on units but do not illustrate controllers and/or sensors.

FIGS. 22-29 illustrates an add on unit 200 that is configured to be attached to a cable via one or more adaptors—such as add on interface 205 and such as clamps 210.

The add on unit includes a top cover 211 (see FIG. 22), a protective filter 201, an apertured element 203 that includes apertured element openings 203′ that is movable upwards and downwards, wherein the sensors space is defined, at least in part, by an apertured inner housing 202 that has fluid openings 202′.

In an open condition—the fluid openings 202′ and the apertured element openings 203′ are aligned. This is illustrated in FIGS. 22, 23 and 24.

In an closed condition—the fluid openings 202′ and the apertured element openings 203′ are misaligned. This is illustrated in FIGS. 14, 26, 27 and 28.

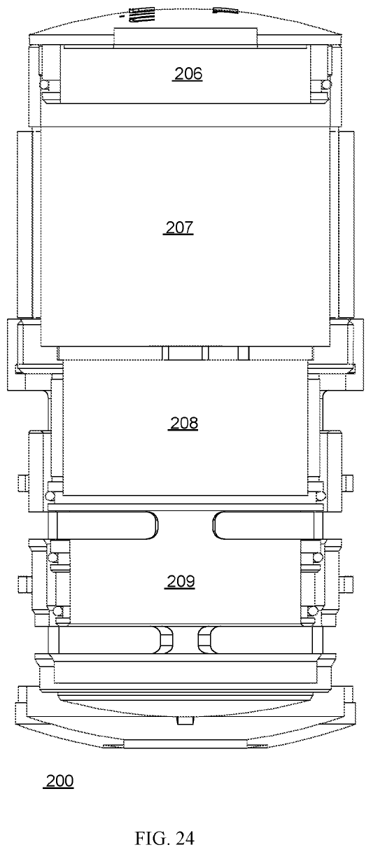

FIG. 22 also illustrated an upper part 204 of the add on unit, FIG. 24 illustrates a cross section of the add on unit that illustrates multiple inner spaces (from top to bottom)—206, 207, 208, 209—of the add on unit—whereas one or more inner spaces may be sealed from each other.

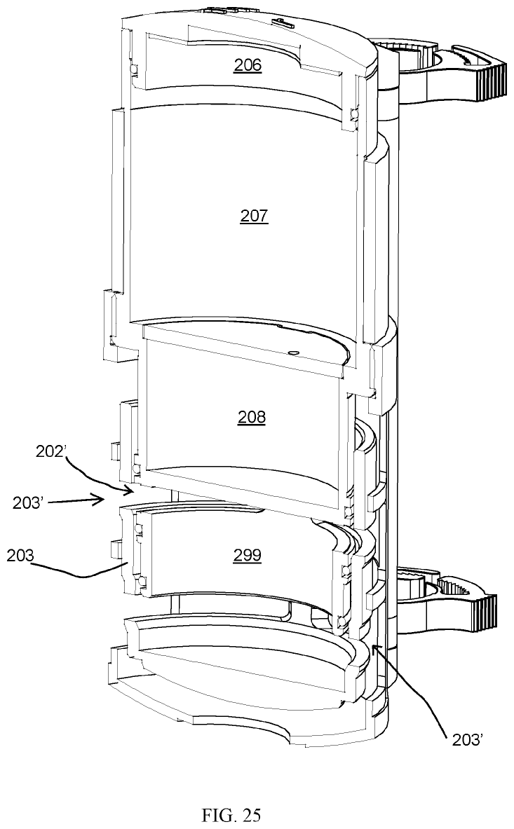

FIG. 25 illustrates a cross section of the add on unit and shows the alignment between the fluid openings 202′ and the apertured element openings 203′.

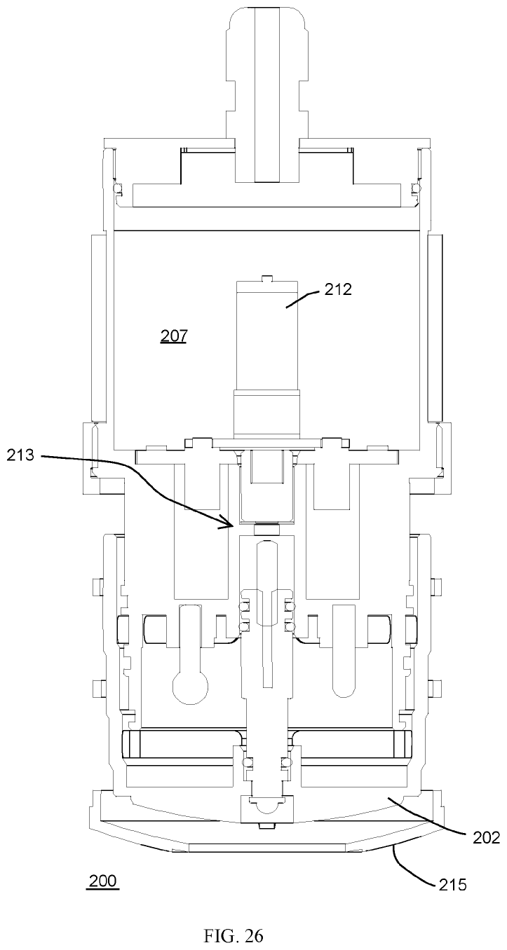

FIG. 26 illustrate a motor 212 located within inner space 207 that moves an axis in a rotational axis that is translated by gear 213 to a upwards or downwards movement of the apertured element 203. FIG. 26 also illustrated the bottom 215 of the add on unit.

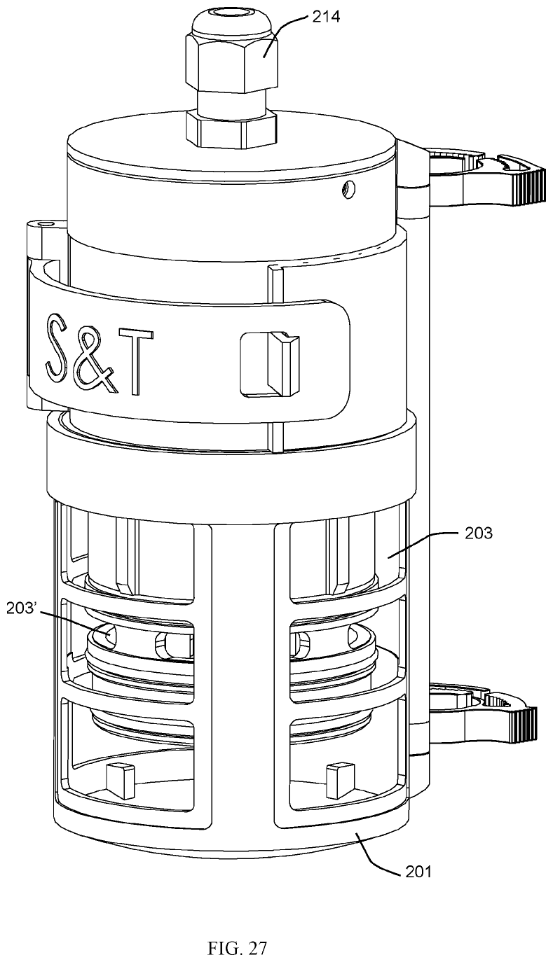

FIG. 27 also illustrates a upper port 214 of the add on unit.

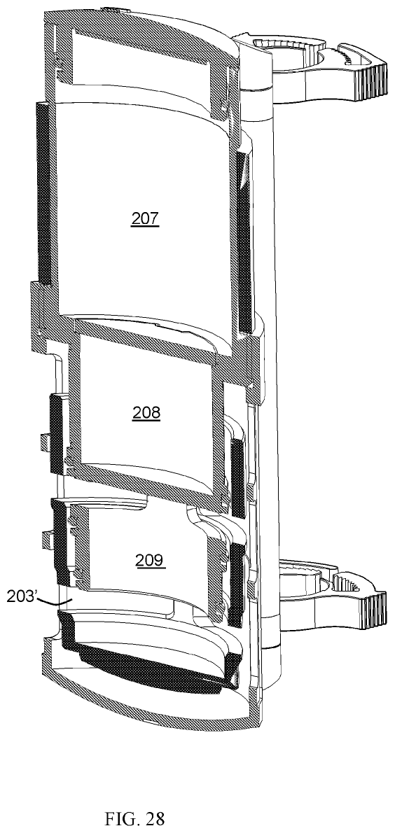

FIG. 28 illustrates some spaces of the add on unit when in the closed position.

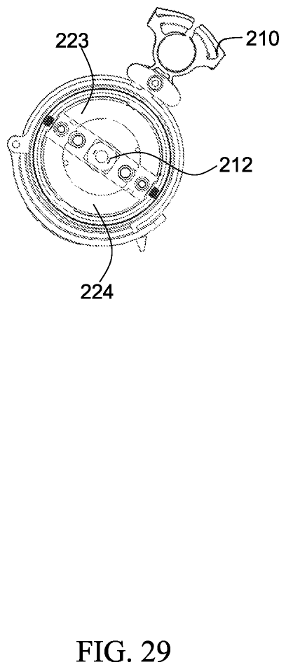

FIG. 29 illustrates a top view of the add on unit where the top cover 211 is removed—exposing a contactless fluid sensor that is a capacitor that has two plates 223 and 224. FIG. 29 also illustrates motor 212 and clamp 210.

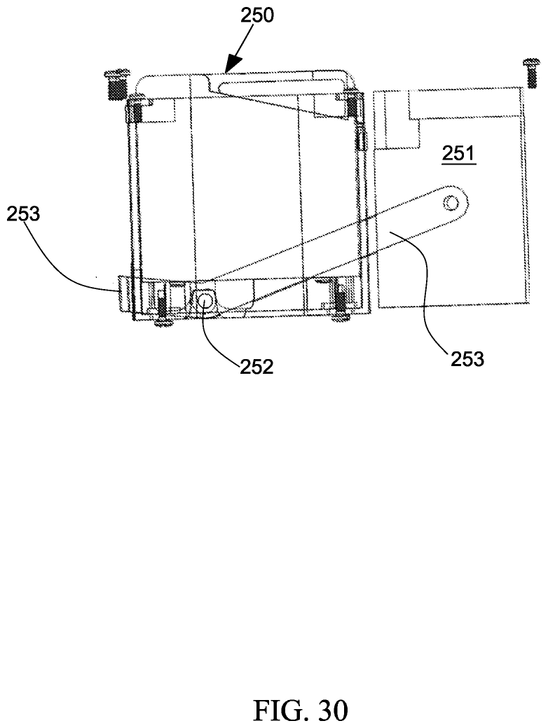

FIGS. 30-33 illustrate a floating unit 251 that is connected via a rotatable element 253 that rotates about axis 252 to move a fluid opening closing unit included in the rotatable element to selectively open or close a fluid opening (now shown) of the add on unit 250.

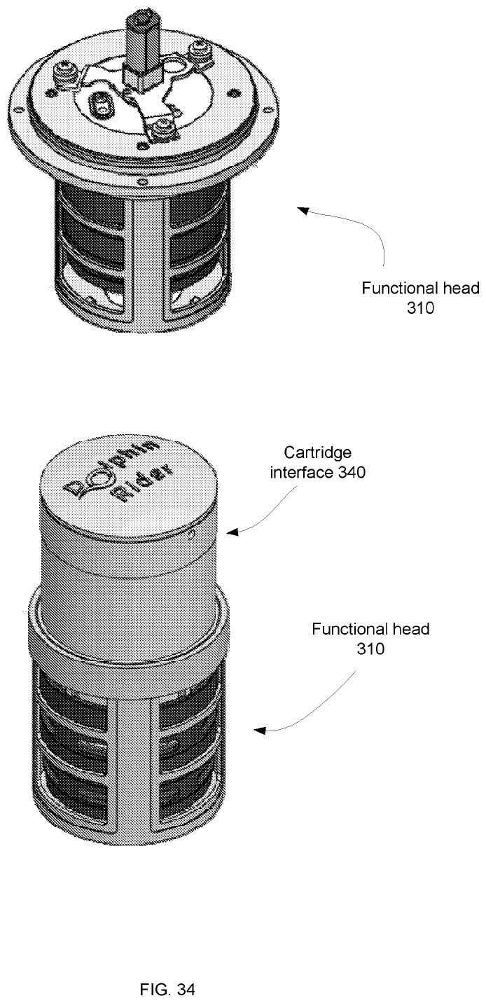

FIGS. 34-38 illustrate examples of an add on unit that includes a functional head, a cartridge interface 340 and a removable and/or replaceable cartridge. The add on unit may be configured to perform alkalinity measurements.

FIG. 34 illustrates a functional head 310 and also illustrates a functional head 310 that is connected to a cartridge interface 340.

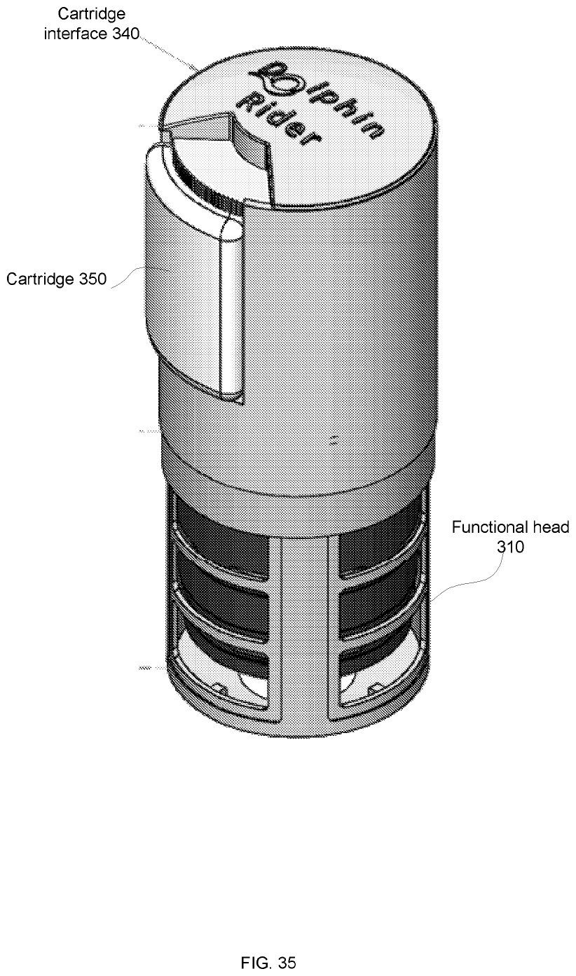

FIG. 35 illustrates a functional head 310 that is connected to a cartridge interface 340 that is detachably connected to cartridge 350.

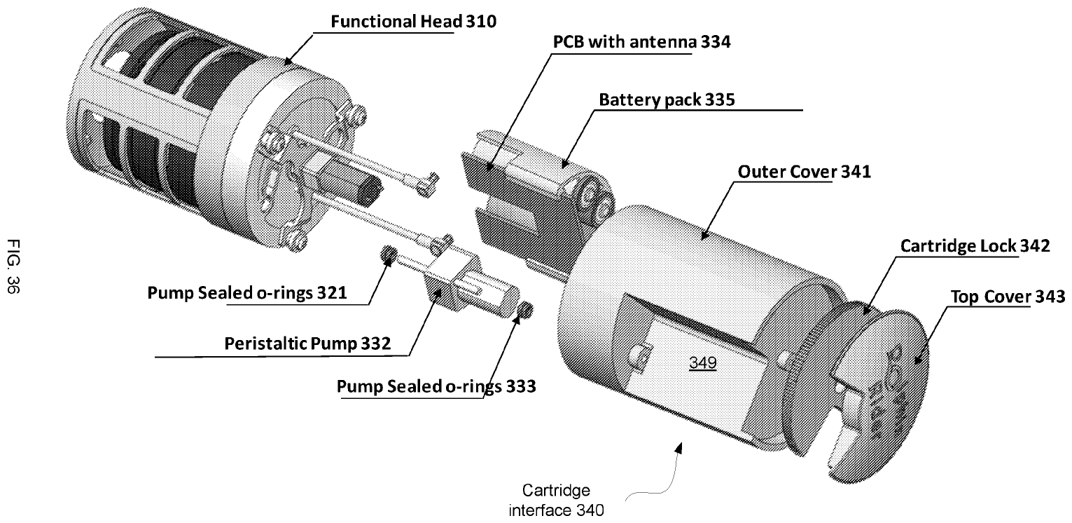

FIG. 36 is an exploded cross sectional view of an add on unit that includes functional head 310, and cartridge interface 340.

Cartridge interface has an outer cover 341, cartridge lock 342, a top cover 343 and inner part 349 that defined a first inner space and contacts a cartridge.

The cartridge interface 341 may also store (via another inner space) a battery pack 335 and electronics such as a printer circuit board (PCB) with antenna 334 and additional electronics (not shown)—such as a controller for controlling the provision of fluid to the measuring cell and/or a processor for processing pH measurements.

A measuring cell (now shown) of the functional head is fluidly coupled to a cartridge via a fluid control unit such as a peristaltic pump 332. FIG. 36 illustrates pump sealing O-Rings 321 and 333 at different ports of the peristaltic pump 332.

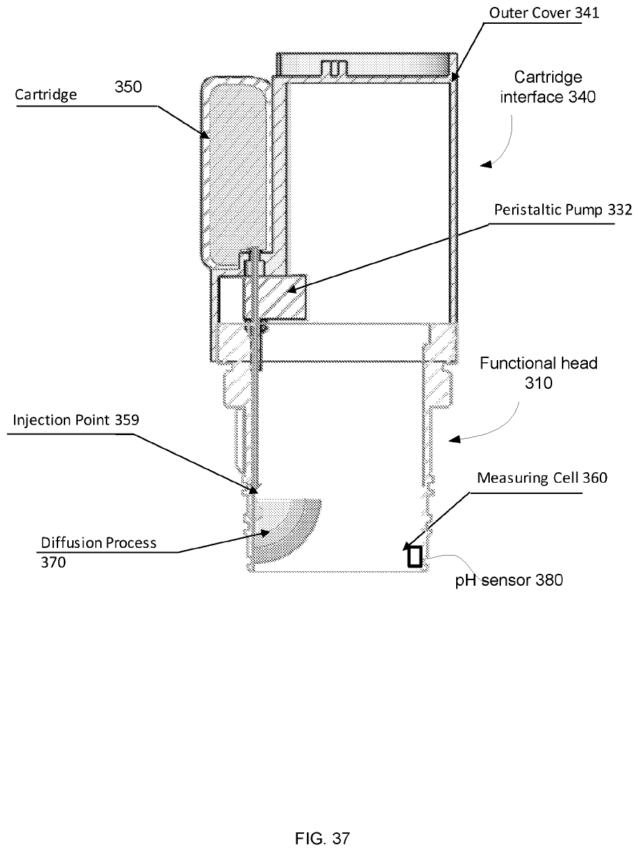

FIG. 37 is a cross sectional view of a functional head 310 that is connected to a cartridge interface 340 (having an outer cover 341) that is detachably connected to cartridge 350. The functional head 310 includes a measuring cell 360 that has an open bottom (or includes at least one fluid opening for receiving the fluid of the pool) or is otherwise exposed to the fluid of the pool. The measuring cell is fluidly coupled to the cartridge 350 via a fluid control unit such as a peristaltic pump 332—or any other flow control unit that is capable of outputting a predetermined amount of cartridge fluid from the cartridge. FIG. 36 illustrates an injection point 359 that outputs the predefined amount of cartridge fluid into the measuring cell 360. The cartridge fluid may be a HCL acid.

The predetermined amount of cartridge fluid may undergo a discussion process 370 or any other mixing or propagating process within the measuring cell 360.

The predetermined amount of cartridge fluid may have a known property—such as a known acidity or a known pH level.

The alkalinity of the fluid of the pool can be measured based on a pH of the fluid (measured by pH sensor 380 of the measuring cell) before (pH_before) the reception of the predetermined amount of cartridge fluid, and after (pH_after) the reception of the predetermined amount of cartridge fluid (the “after” measurement is executed after a predefined propagation or mixing of diffusion period of the predetermined amount of cartridge fluid in the fluid within the measuring cell 360).

For example: Alkalinity (of the fluid of the pool)=(pH_before−pH_after)*Factor.

The value of the Factor is known—for example based on experiments, based on the concentration of the cartridge fluid within the measuring cell, and the like.

A larger amount of HCL acid—in comparison to the amount of HCL acid used for measurement of alkalinity—may be used for cleaning the pH sensor and/or for cleaning any electrode within the measuring cell.

An addition of pH and ORP buffer may be used for calibrating the pH sensor.

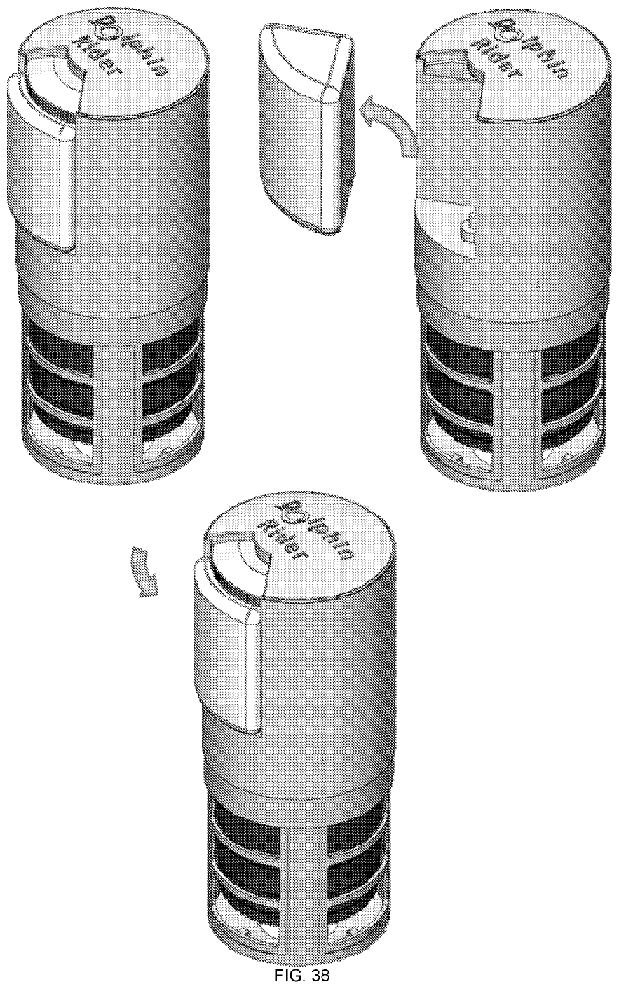

FIG. 38 illustrates an example of replacing a used cartridge by a new cartridge. The removal of the used cartridge includes rotating a cartridge lock (denoted 342 in FIG. 36) at a first rotational direction. The locking of the new cartridge includes rotating the cartridge lock at an opposite rotations direction.

Any reference to any one of “including” or “comprising” or “having” may be applied mutatis mutandis to any one of “consisting” and “consisting essentially of”. For example—any method may include at least the steps included in the figures and/or in the specification, only the steps included in the figures and/or the specification. The same applies to the pool cleaning robot and the mobile computer.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

In the foregoing specification, the invention has been described with reference to specific examples of embodiments of the invention. It will, however, be evident that various modifications and changes may be made therein without departing from the broader spirit and scope of the invention as set forth in the appended claims.

Moreover, the terms “front,” “back,” “top,” “bottom,” “over,” “under” and the like in the description and in the claims, if any, are used for descriptive purposes and not necessarily for describing permanent relative positions. It is understood that the terms so used are interchangeable under appropriate circumstances such that the embodiments of the invention described herein are, for example, capable of operation in other orientations than those illustrated or otherwise described herein.

Those skilled in the art will recognize that the boundaries between logic blocks are merely illustrative and that alternative embodiments may merge logic blocks or circuit elements or impose an alternate decomposition of functionality upon various logic blocks or circuit elements. Thus, it is to be understood that the architectures depicted herein are merely exemplary, and that in fact many other architectures can be implemented which achieve the same functionality.

Any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality.

Furthermore, those skilled in the art will recognize that boundaries between the above described operations merely illustrative. The multiple operations may be combined into a single operation, a single operation may be distributed in additional operations and operations may be executed at least partially overlapping in time. Moreover, alternative embodiments may include multiple instances of a particular operation, and the order of operations may be altered in various other embodiments.

Also for example, in one embodiment, the illustrated examples may be implemented as circuitry located on a single integrated circuit or within a same device. Alternatively, the examples may be implemented as any number of separate integrated circuits or separate devices interconnected with each other in a suitable manner.

Also for example, the examples, or portions thereof, may implemented as soft or code representations of physical circuitry or of logical representations convertible into physical circuitry, such as in a hardware description language of any appropriate type.

Also, the invention is not limited to physical devices or units implemented in non-programmable hardware but can also be applied in programmable devices or units able to perform the desired device functions by operating in accordance with suitable program code, such as mainframes, minicomputers, servers, workstations, personal computers, notepads, personal digital assistants, electronic games, automotive and other embedded systems, cell phones and various other wireless devices, commonly denoted in this application as ‘computer systems’.

However, other modifications, variations and alternatives are also possible. The specifications and drawings are, accordingly, to be regarded in an illustrative rather than in a restrictive sense.

In the claims, any reference signs placed between parentheses shall not be construed as limiting the claim. The word ‘comprising’ does not exclude the presence of other elements or steps then those listed in a claim. Furthermore, the terms “a” or “an,” as used herein, are defined as one as or more than one. Also, the use of introductory phrases such as “at least one” and “one or more” in the claims should not be construed to imply that the introduction of another claim element by the indefinite articles “a” or “an” limits any particular claim containing such introduced claim element to inventions containing only one such element, even when the same claim includes the introductory phrases “one or more” or “at least one” and indefinite articles such as “a” or “an.” The same holds true for the use of definite articles. Unless stated otherwise, terms such as “first” and “second” are used to arbitrarily distinguish between the elements such terms describe. Thus, these terms are not necessarily intended to indicate temporal or other prioritization of such elements the mere fact that certain measures are recited in mutually different claims does not indicate that a combination of these measures cannot be used to advantage.

Any system, apparatus or device referred to this patent application includes at least one hardware component.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

We claim:

1. An add on unit, comprising:
a first communication unit that is configured to communicate with a pool related platform;
a second communication unit that is configured to communicate with a computerized system;
one or more additional components;
a housing;
one or more adaptors that are configured to connect the add on unit to at least one out of the pool related platform or a cable that is connected to the pool related platform.

2. The add on unit according to claim 1, wherein the one or more adaptors are configured to connect the add on unit to the at least one out of the pool related platform or the cable.

3. The add on unit according to claim 1, wherein the one or more adaptors are self-connectable to the at least one out of the pool related platform or the cable.

4. The add on unit according to claim 1, wherein the one or more adaptors comprise one or more cable clip adaptors.

5. The add on unit according to claim 1, wherein the housing is a sealed housing.

6. The add on unit according to claim 1, comprising a power supply.

7. The add on unit according to claim 1, wherein the add on unit is shaped and sized to be at least partially inserted in a filtering unit of the pool related platform.

8. The add on unit according to claim 1, wherein the add on unit is shaped and sized to be located within a housing of the pool related platform and to be connected to a filtering unit of the pool related platform.

9. The add on unit according to claim 1, wherein the add on unit is shaped and sized to be located within a housing of the pool related platform and to be connected to a motor unit of the pool related platform.

10. The add on unit according to claim 1, wherein the one or more adaptors are configured to connect the add on unit to the pool related platform.

11. The add on unit according to claim 1, wherein the one or more adaptors are configured to connect the add on unit to the cable.

12. The add on unit according to claim 1, comprising a navigation unit, wherein an additional element is a sensor that belongs to the navigation unit.

13. The add on unit according to claim 1, comprising a drown detection unit, wherein an additional element is a sensor that belongs to the drown detection unit.

14. The add on unit according to claim 1, wherein an additional element is a water quality sensor.

15. The add on unit according to claim 1, wherein an additional element is an image sensor.

16. The add on unit according to claim 1, wherein an additional element is a i microphone.

17. The add on unit according to claim 1, wherein an additional element is an accelerometer.

18. The add on unit according to claim 1, wherein an additional element is a temperature sensor.

19. The add on unit according to claim 1, wherein an additional element is a chemical agent distribution unit.

20. The add on unit according to claim 1, wherein an additional element is a removable floating element.

21. The add on unit according to claim 1 wherein the first communication unit is a first wireless communication unit and the second communication unit is a wireless communication unit.

22. The add on unit according to claim 1, wherein the one or more adaptors are configured to detachably connect the add on unit to the at least one out of the pool related platform or the cable.

23. The add on unit according to claim 1, wherein the one or more adaptors are configured to one time connect the add on unit to the at least one out of the pool related platform or the cable.

24. A method for operating an add on unit of any one of the preceding claims.

25. A sensor protective system, comprising:
a sensing unit that comprises one or more sensing regions for sensing one or more parameters of a fluid when in contact with the fluid; and
a protection unit that is configured to prevent the one or more sensing regions from being damaged due to dryness.

26. The sensor protective system according to claim 25, wherein the protection unit is configured to stop a measurement of at least one of the one or more parameters of the fluid.

27. The sensor protective system according to claim 26, wherein the protection unit is configured to stop the measurement when detecting a triggering event that indicates that a completion of the measurement will introduce a dryness related damage.

28. The sensor protective system according to claim 25, wherein the protection unit is configured to perform a protective step following a detection of a triggering event.

29. The sensor protective system according to claim 28, wherein the triggering event is an extraction of the sensor protective system from the fluid.

30. The sensor protective system according to claim 28, wherein the triggering event comprises having the sensor protective system tilted at an angle that is outside a predefined angular range.

31. The sensor protective system according to claim 28, wherein the detection of the triggering event is based on acceleration signals.

32. The sensor protective system according to claim 28, wherein the detection of the triggering event is based on tilt angle signals.

33. The sensor protective system according to claim 28, wherein the detection of the triggering event is based on sensing an environment of the add on unit.

34. The sensor protective system according to claim 33 comprising a contactless fluid sensor that is configured to sense a wetness of the environment without contacting the environment.

35. The sensor protective system according to claim 25, wherein the one or more sensing regions are located within one or more spaces of the add on unit.

36. The sensor protective system according to claim 35, wherein the protection unit is configured to maintain at least a predefined humidity within the one or more space.

37. The sensor protective system according to claim 35, wherein the protection unit is configured to maintain at least a predefined amount of fluid within the one or more spaces.

38. The sensor protective system according to claim 35, wherein the protection unit is configured to maintain the one or more sensing regions within fluid captured within the one or more space.

39. The sensor protective system according to claim 35, wherein the protection unit is configured to control a flow of fluid from the one or more spaces.

40. The sensor protective system according to claim 35, wherein the protection unit is configured to control a flow of fluid to the one or more spaces.

41. The sensor protective system according to claim 35, wherein the protection unit comprises one or more flow control elements that control a flow of fluid between the one or more spaces and an environment of the one or more spaces.

42. The sensor protective system according to claim 41, wherein the one or more flow control units comprise one or more pumps.

43. The sensor protective system according to claim 41, wherein the one or more flow control units comprise one or more floating units.

44. The sensor protective system according to claim 41, wherein the one or more flow control units comprise one or more motors.

45. The sensor protective system according to claim 41, wherein the one or more spaces comprises one or more fluid openings, and wherein the one or more flow control units are configured to control a flow of the fluid through the one or more fluid openings.

46. The sensor protective system according to claim 45, wherein the one or more flow control units comprises one or more movable seals that are configured to selectively seal the one or more fluid openings.

47. The sensor protective system according to claim 45, wherein the one or more flow control units comprises one or more fluid openings closing elements.

48. The sensor protective system according to claim 45, wherein the one flow control units comprise one or more apertured elements that are configured to be moved between (a) an open position in which apertures of the apertured elements are at least partially aligned with the one or more fluid openings, and (b) a closed position in which apertures of the apertured elements misaligned with the one or more fluid openings and the apertured elements and block a passage of fluid between the one or more spaces and an environment of the one or more spaces.

49. The sensor protective system according to claim 25 comprising one or more adaptors that are configured to connect the sensor protective system to at least one out of a pool related platform or a cable that is connected to the pool related platform.

50. The sensor protective system according to claim 49, wherein the one or more adaptors are configured to detachably connect the sensor protective system to the at least one out of the pool related platform or the cable.

51. The sensor protective system according to claim 49, wherein the one or more adaptors are self-connectable to the at least one out of the pool related platform or the cable.

52. The sensor protective system according to claim 49, wherein the one or more adaptors comprise one or more cable clip adaptors.

53. The sensor protective system according to claim 25, comprising a power supply.

54. The sensor protective system according to claim 25, wherein the sensor protective system is shaped and sized to be at least partially inserted in a filtering unit of the pool related platform.

55. The sensor protective system according to claim 25, wherein the sensor protective system is shaped and sized to be located within a housing of the pool related platform and to be connected to a filtering unit of the pool related platform.

56. The sensor protective system according to claim 25, wherein the one or more sensors comprise water quality sensor.

57. The sensor protective system according to any claim of claims 25-56 wherein the sensor protective system is an add on unit.

58. The sensor protective system according to any claim of claims 25-56 wherein the preceding claims wherein the sensor protective system differs from an add on unit.

59. A method for operating an sensor protective system of any claim of claims 25-56.
